(19) United States
(12) Patent Application Publication
(10) Pub. No.: US 2026/0139002 A1
(43) Pub. Date: May 21, 2026

(54) PURIFICATION OF POLYPEPTIDES

(71) Applicant: Adonis Health Inc., Washington DC, DC (US)

(72) Inventors: Darr R. Kadlubowski, Phoenix, AZ (US); Cyrille V. Gineste, Merritt Island, FL (US)

(73) Assignee: Adonis Health Inc., Washington DC, DC (US)

(21) Appl. No.: 19/246,022

(22) Filed: Jun. 23, 2025

Related U.S. Application Data
(60) Provisional application No. 63/723,490, filed on Nov. 21, 2024.

(51) Int. Cl. C07K 1/34; B01D 15/16; B01D 15/32; B01D 15/34; B01D 69/02; C07K 1/16; C07K 1/20

(52) U.S. Cl. CPC C07K 1/34; B01D 15/166; B01D 15/325; B01D 15/34; B01D 69/02; C07K 1/16; C07K 1/20; B01D 2325/34

(57) ABSTRACT

Purification of a peptide is described herein.

### PRIORITY CLAIM

This application claims priority to U.S. Provisional Patent Application No. 63/723,490, filed Nov. 21, 2024, which is incorporated by reference in its entirety.

### TECHNICAL FIELD

This invention relates to purification of peptides.

### BACKGROUND

Peptides can be challenging to synthesize due to expense and time. Thus, developing methods of purifying peptides can be economically valuable.

### SUMMARY

The present invention features a method of purifying a peptide.

In one aspect, a method for purifying a peptide can include suspending a powder including the peptide and one or more of a matrix polymer, a carboxylate salt, and a cellulose in a polar liquid, filtering the suspension to yield a target filtrate, purifying the target filtrate by one or more of tangential flow filtration, size exclusion chromatography, reverse phase liquid chromatography, or combinations thereof, and collecting the purified peptide.

In certain circumstances, the polar liquid can include water, methanol, ethanol, a propanol, or a butanol.

In certain circumstances, the polar liquid can be ethanol, and the method can include suspending an intermediate residue in water to form a second suspension and filtering the second suspension to produce the target filtrate.

In certain circumstances, the polar liquid can be water.

In certain circumstances, purifying the target filtrate can include tangential flow filtration to yield a residual solution. In certain circumstances, the residual solution can be further purified by high-performance liquid chromatography (HPLC), for example, isocratic HPLC or gradient HPLC. Alternatively, the residual solution can be further purified by size exclusion chromatography.

In certain circumstances, the powder includes an absorption enhancer.

In certain circumstances, purifying the target filtrate can include size exclusion chromatography to yield a residual solution. In certain circumstances, the residual solution can be further purified by high-performance liquid chromatography, for example, isocratic HPLC or gradient HPLC.

In certain circumstances, purifying the target filtrate includes isocratic HPLC to yield a residual solution. In certain circumstances, the residual solution can be further purified by gradient HPLC.

In certain circumstances, purifying the target filtrate can include gradient HPLC to yield a residual solution. In certain circumstances, the residual solution can be further purified by isocratic HPLC.

In certain circumstances, the peptide can be semaglutide.

In certain circumstances, the absorption enhancer can include sodium 8-(2-hydroxybenzamido)octanoate).

In certain circumstances, the matrix polymer can include polyvinylpyrrolidone.

In certain circumstances, the cellulose can include microcrystalline cellulose.

In certain circumstances, the carboxylate salt can include magnesium stearate.

In certain circumstances, the powder can be a crushed or pulverized tablet.

In certain circumstances, the purified polypeptide is in its free base form.

In certain circumstances, the purified polypeptide is in a salt free form.

In certain circumstances, the purified polypeptide is in its free acid form.

Other aspects, embodiments, and features as disclosed herein will be apparent from the following description, the drawings, and the claims.

### BRIEF DESCRIPTION OF THE DRAWINGS

FIG. 1 shows a schematic of a method of purifying a peptide.

FIG. 2 shows a detailed schematic of a method of a first step of purifying a peptide.

FIG. 3 shows a detailed schematic of a method of a second step of purifying a peptide.

FIG. 4 shows a detailed schematic of methods of a second step of purifying a peptide.

FIG. 5 shows a detailed experiment of a first step of purifying a peptide.

FIG. 6 shows flow parameters of a second step of purifying a peptide.

FIG. 7 shows a chromatogram of purifying a peptide.

FIG. 8 shows flow parameters of a second step of purifying a peptide.

FIG. 9 shows a chromatogram of purifying a peptide.

FIG. 10 shows a chromatogram of purifying a peptide.

FIG. 11 shows chromatograms of increasing concentrations of acetic acid in water.

FIG. 12 shows chromatograms of increasing concentrations of trifluoroacetic acid in water.

FIG. 13 shows a chromatogram of semaglutide chemically synthesized and cleaved from its resin using trifluoroacetic acid.

FIG. 14 shows a chromatogram of semaglutide obtained through purification from an FDA-approved, clinically available oral tablet form of semaglutide.

FIG. 15 shows body weight in DIO mice (grams).

FIG. 16 shows percentage change of body weight in DIO mice (grams).

FIG. 17 shows food intake in DIO mice (grams).

FIG. 18 shows blood glucose after 6 hrs fasting in DIO mice.

FIGS. 19A-19C shows the progressive purification of semaglutide using tangential flow filtration (TFF). FIG. 19A shows the first concentrated solution after the first TFF round. FIG. 19B shows the third concentrated solution after the third TFF round. FIG. 19C shows the fifth concentrated solution after the fifth TFF round.

### DETAILED DESCRIPTION

It is to be appreciated that certain aspects, modes, embodiments, variations, and features of the present methods and compositions are described below in various levels of detail in order to provide a substantial understanding of the present disclosure.

Large-scale economic purification of peptides is an increasingly important issue for the biotechnology industry. The separation of the peptide of interest from other impurities can be accomplished using a combination of different chromatographic techniques. These techniques separate mixtures of peptides based on their charge, degree of hydrophobicity, size, or specific interaction between the peptide of interest and the immobilized capture material. Various chromatographic resins are available for each of these techniques, allowing precise customization of the purification scheme for the particular peptide involved. The essence of these separation methods is that the peptide moves at different speeds along the column, achieving physical separation as they pass through, or selectively adheres to the separation medium and is eluted differently. In some cases, the peptide of interest is separated from impurities when the impurities specifically adhere to the column while the peptide of interest does not.

As discussed herein, a method for purifying a peptide from a composition comprising the peptide and at least one contaminant can include a coarse purifying step and a fine purifying step. The coarse purifying step can include suspension and filtration of a contaminated powder including the peptide to yield a solution (or target filtrate) including the peptide. In some embodiments, after the first suspension comprising the contaminated powder and a polar liquid is filtered, an intermediate residue is obtained, for instance when the first suspension is generated using ethanol as the polar liquid, that includes the peptide. This is followed by a second suspension of the intermediate residue in water prior to another filtration step to produce the solution including the peptide. The fine purifying step follows the coarse purifying step to yield a residual solution including a purified form of the peptide. The fine purifying step can be a chromatography step or tangential flow filtration (TFF) step, or combinations thereof.

The term “peptide” refers to a sequence of two or more amino acid residues linked by peptide bonds. The peptide may be linear or cyclic, and it may be composed of L-amino acids, D-amino acids, or a combination thereof. The term encompasses native peptides as well as peptides that are modified or derivatized by one or more chemical substituents, whether naturally occurring or synthetically introduced. The peptide may include modifications at the N-terminus, C-terminus, side chains, backbone, or internal residues, and may optionally be conjugated to non-peptidic moieties to improve stability, activity, or pharmacokinetic properties.

The term “polypeptide,” which sometimes can be used to describe a peptide that has a chain length and properties sufficient to create a higher level of tertiary and/or quaternary structure, is included herein under the term “peptide.” Typically, the peptide will have a molecular weight of at least about 3 kDa to 20 kDa, in some preferrable embodiments 3 kDa to 6 kDa. In certain embodiments, the peptide can be an incretin, for example, semaglutide. A “purified” peptide is one that exists in a more pure form than initially synthesized or amplified under laboratory conditions. Purity is a relative term and does not necessarily mean absolute purity. In some embodiments, purity can be at least 90%, at least 95%, or at least 98%. In certain embodiments, purity can be at least 98% purity.

The term “absorption enhancer” refers to a first compound that increases the rate and/or the extent of absorption of a drug or second compound across a biological membrane or barrier such as the intestinal lining. The absorption enhancer can augment or facilitate the transport of the drug or second compound across the biological barrier (e.g., membrane) in various ways, for instance by temporarily modifying the barrier properties, increasing solubility, or altering drug partitioning to improve bioavailability.

The term “intermediate residue” refers to a solid or semi-solid including a desired peptide that remains after filtration of a first suspension generated by a powder containing said peptide and excipients in a polar liquid such as ethanol during a coarse purifying step.

The term “target filtrate” refers to a solution including a desired peptide in water that is the final output of a coarse purifying step. The target filtrate is the input for the fine purifying step that includes the desired peptide.

The term “residual solution” refers to a solution including a purified form of a desired peptide that has undergone one or more fine purifying steps. The fine purifying step can include one or more chromatography steps, tangential flow filtration (TFF) steps, or combinations thereof.

The term “excipient” refers to pharmaceutically acceptable substances that are combined with an active pharmaceutical ingredient in the formulation of a drug product, but are not themselves therapeutically active.

The term “free base” or “free base form” of a peptide refers to a peptide in which basic functional groups (including the N-terminus) are in their unprotonated (neutral) form, and no acid counter-ions are present such as a trifluoroacetate anion, acetate anion, chloride anion, or other anions.

The term “free acid” or “free acid form” of a peptide refers to a peptide in which acidic functional groups (including the C-terminus) are in their protonated (neutral) form, and no basic counter-ions are present to form a salt.

The term “salt free” or “salt free form” of a peptide refers to a peptide preparation that contains no added counter-ions or residual salts, such as trifluoroacetate, acetate, hydrochloride, or other ionic species.

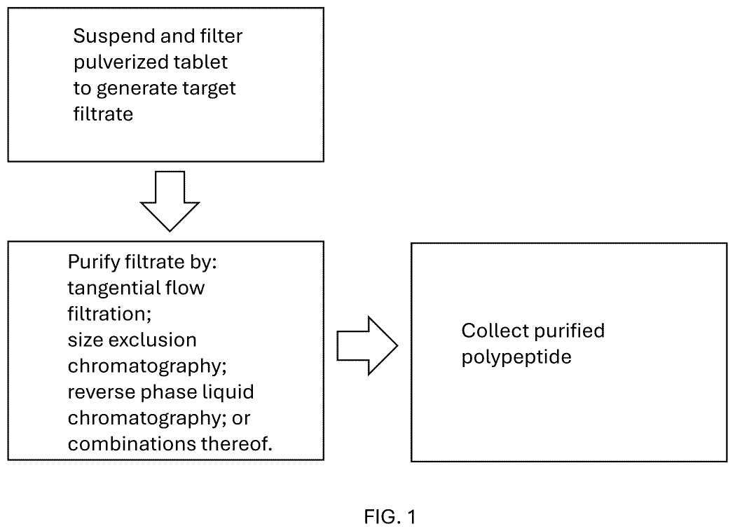

In one aspect, a method for purifying a peptide can include suspending a powder including the peptide and one or more of a matrix polymer, a carboxylate salt, and a cellulose in a polar liquid, filtering the suspension to yield a target filtrate, purifying the target filtrate by one or more of tangential flow filtration (TFF), size exclusion chromatography (SEC), reverse phase liquid chromatography, or combinations thereof, and collecting the purified peptide. In some embodiments, the powder includes an absorption enhancer. The steps are shown in FIG. 1.

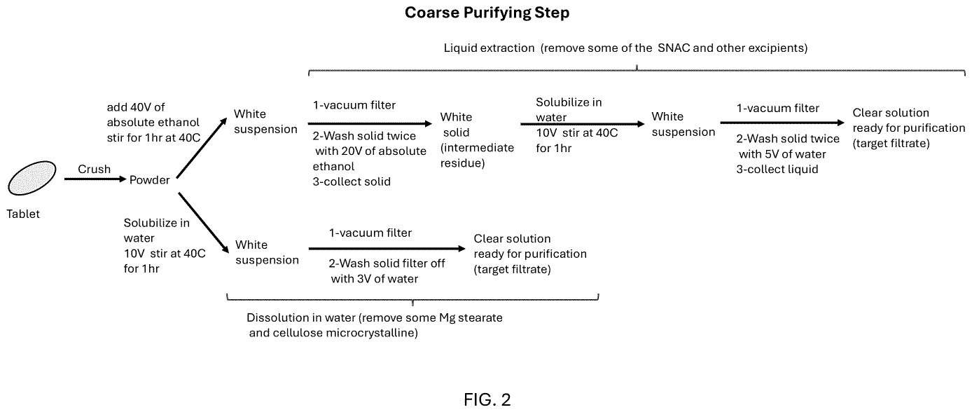

Referring to FIG. 2, a coarse purifying step is shown. In one example, a powder, such as a crushed or pulverized tablet, is processed. The tablet can include the peptide and one or more of an absorption enhancer, such as salcaprozate sodium (SNAC; sodium 8-(2-hydroxybenzamido)octanoate), a cellulose, such as microcrystalline cellulose, a carboxylate salt, such as magnesium stearate, and a matrix polymer, such as polyvinylpyrrolidone (PVP). In certain circumstances, the peptide can be semaglutide.

The peptide and impurities can be purified in a first step by suspension and filtration in a polar liquid. In certain circumstances, the polar liquid can include water, methanol, ethanol, a propanol, or a butanol. In certain circumstances, the polar liquid can be water.

Referring to FIG. 2, the tablet can be crushed and solubilized in absolute ethanol. This can form a suspension that can be stirred, for example, at 40° C. for 1 hour. The suspension can then be filtered, for example, vacuum filtered and the solid washed up to two times with ethanol. The solid can be collected as an intermediate residue. Next, the solid can be solubilized in water. This can form a suspension that is then stirred, for example, at 40° C. for 1 hour, and the final suspension can be filtered to produce a target filtrate or solution. The resulting clear solution (or target filtrate) can move on to one or more further purification steps in the fine purifying step. The clear solution can contain lower amounts of excipients, for example, the absorption enhancer, such as salcaprozate sodium (SNAC; sodium 8-(2-hydroxybenzamido)octanoate), the cellulose, such as microcrystalline cellulose, the carboxylate salt, such as magnesium stearate, or the matrix polymer, such as polyvinylpyrrolidone (PVP).

In an alternative approach, also outlined in FIG. 2, the tablet can be crushed and solubilized in water. This can form a suspension that can be stirred, for example, at 40° C. for 1 hour. The suspension can then be filtered, for example, vacuum filtered and the solid washed with water to produce a solution or target filtrate. The resulting clear solution (or target filtrate) can move on to one or more further purification steps. The clear solution can contain lower amounts of excipients, for example, the absorption enhancer, such as salcaprozate sodium (SNAC; sodium 8-(2-hydroxybenzamido)octanoate), the cellulose, such as microcrystalline cellulose, the carboxylate salt, such as magnesium stearate, or the matrix polymer, such as polyvinylpyrrolidone (PVP).

In certain circumstances, the polar liquid can be ethanol, and the method can include suspending an intermediate residue in water to form a second suspension and filtering the second suspension to produce the target filtrate containing the peptide.

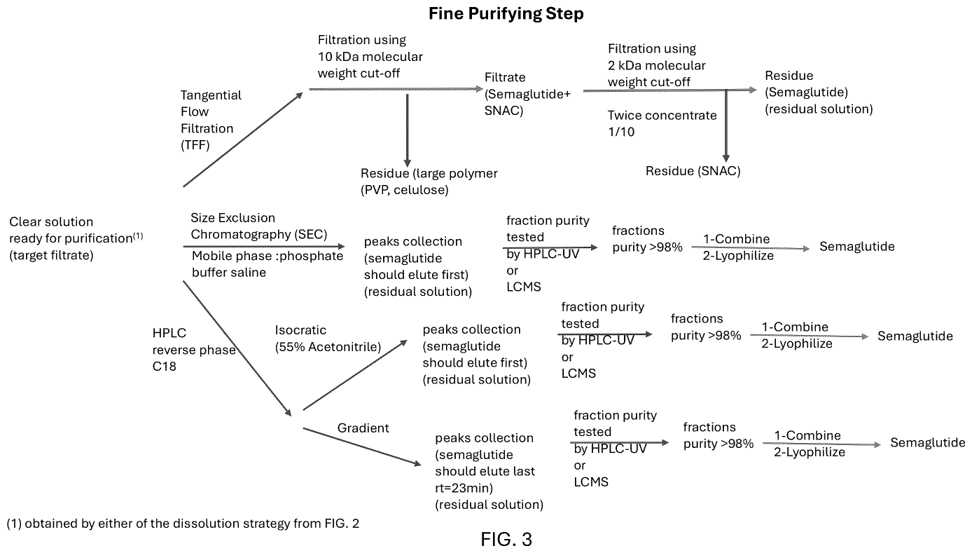

Referring to FIG. 3, a fine purifying step is shown. Purifying the target filtrate can include tangential flow filtration (TFF) to yield a residual solution containing the peptide. In certain circumstances, the residual solution can be further purified by high-performance liquid chromatography (HPLC), for example, isocratic HPLC or gradient HPLC. Alternatively, the residual solution can be further purified by size exclusion chromatography (SEC).

In certain circumstances, purifying the target filtrate can include size exclusion chromatography (SEC) to yield a residual solution. In certain circumstances, the residual solution can be further purified by high-performance liquid chromatography (HPLC), for example, isocratic HPLC or gradient HPLC.

In certain circumstances, purifying the target filtrate includes isocratic HPLC to yield a residual solution. In certain circumstances, the residual solution can be further purified by gradient HPLC.

In certain circumstances, purifying the target filtrate can include gradient HPLC to yield a residual solution. In certain circumstances, the residual solution can be further purified by isocratic HPLC.

Referring to FIG. 3, in one example, tangential flow filtration (TFF) can be used to filter the clear solution (or target filtrate) using 10 kDa molecular weight cut-off, which can remove or reduce the presence of residual large polymer PVP and/or cellulose. The filtrate, which can include the peptide and SNAC, can then be filtered using 2 kDa molecular weight cut-off, which can remove the residual SNAC. The resulting concentrate can include the residual peptide in a residual solution.

In some embodiments, the clear solution (or target filtrate) is first processed through five or less rounds of TFF using a 10 kDa molecular weight cut-off before being further processed through ten or less rounds of TFF using a 2 kDa molecular weight cut-off. In some embodiments, the clear solution (or target filtrate) is only processed through ten or less rounds of TFF using a 2 kDa molecular weight cut-off.

In some embodiments, the clear solution (or target filtrate) is first processed through five rounds, less than five rounds, less than four rounds, less than three rounds, less than two rounds, or one round of TFF using a 10 kDa molecular weight cut-off before being further processed through ten rounds, less than ten rounds, less than nine rounds, less than eight rounds, less than seven rounds, less than six rounds, less than five rounds, less than four rounds, less than three rounds, less than two rounds, or one round of TFF using a 2 kDa molecular weight cut-off.

In some embodiments, the clear solution (or target filtrate) is only processed through ten rounds, less than ten rounds, less than nine rounds, less than eight rounds, less than seven rounds, less than six rounds, less than five rounds, less than four rounds, less than three rounds, less than two rounds, or one round of TFF using a 2 kDa molecular weight cut-off.

The amount of starting material or powder containing a target peptide such as semaglutide and additional excipients, as well as the size of the TFF cassettes being used, can determine whether TFF with a 10 kDa molecular weight cut-off should be used prior to TFF with a 2 kDa molecular weight cut-off or just TFF with a 2 kDa molecular weight cut-off alone, as well as how many rounds of TFF should be performed at each 10 kDa molecular weight cut-off or 2 kDa molecular weight cut-off step, to achieve a desired peptide purity.

In some embodiments, the TFF cassette can range from about 0.01 m² to about 60 m²

In some embodiments, the TFF cassette can be about 0.01 m², about 0.02 m², about 0.03 m², about 0.04 m², about 0.05 m², about 0.06 m², about 0.07 m², about 0.08 m², about 0.09 m², about 0.1 m², about 0.11 m², about 0.12 m², about 0.13 m², about 0.14 m², about 0.15 m², about 0.16 m², about 0.17 m², about 0.18 m², about 0.19 m², about 0.2 m², about 0.25 m², about 0.3 m², about 0.35 m², about 0.4 m², about 0.45 m², about 0.5 m², about 0.55 m², about 0.6 m², about 0.65 m², about 0.7 m², about 0.75 m², about 0.8 m², about 0.85 m², about 0.9 m², about 0.95 m², about 1.0 m², about 1.1 m², about 1.2 m², about 1.3 m², about 1.4 m², about 1.5 m², about 1.6 m², about 1.7 m², about 1.8 m², about 1.9 m², about 2.0 m², about 2.1 m², about 2.2 m², about 2.3 m², about 2.4 m², about 2.5 m², about 2.6 m², about 2.7 m², about 2.8 m², about 2.9 m², about 3.0 m², about 3.1 m², about 3.2 m², about 3.3 m², about 3.4 m², about 3.5 m², about 3.6 m², about 3.7 m², about 3.8 m², about 3.9 m², about 4.0 m², about 4.1 m², about 4.2 m², about 4.3 m², about 4.4 m², about 4.5 m², about 4.6 m², about 4.7 m², about 4.8 m², about 4.9 m², about 5.0 m², about 5.5 m², about 6.0 m², about 6.5 m², 7.0 m², about 7.5 m², about 8.0 m², about 8.5 m², about 9.0 m², about 9.5 m², about 10.0 m², about 11.0 m², about 12.0 m², about 13.0 m², about 14.0 m², about 15.0 m², about 16.0 m², about 17.0 m², about 18.0 m², about 19.0 m², about 20.0 m², about 25.0 m², about 30.0 m², about 35.0 m², about 40.0 m², about 45.0 m², about 50.0 m², about 55.0 m², or about 60.0 m².

In another example, size exclusion chromatograph (SEC), for example, with a mobile phase phosphate buffer saline (25 mM phosphate buffer, 100 mM KCl, 10% acetonitrile pH 7.00), can be used to purify the clear solution (or target filtrate). In the SEC, the peptide elutes first. The fraction can be tested for purity by HPLC-UV or liquid chromatography-mass spectrometry (LC-MS). The fraction purity can be greater than 98%. The fractions can be combined and lyophilized to yield the purified peptide.

In some embodiments, the fraction purity can be at least 95%.

In some embodiments, the fraction purity can be at least 95%, at least 96%, at least 97%, at least 98%, at least 99%, or greater than 99%.

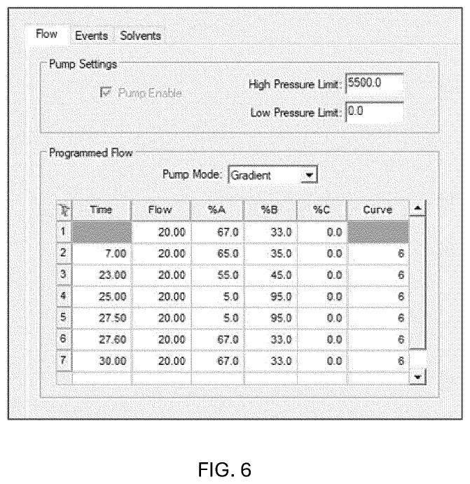

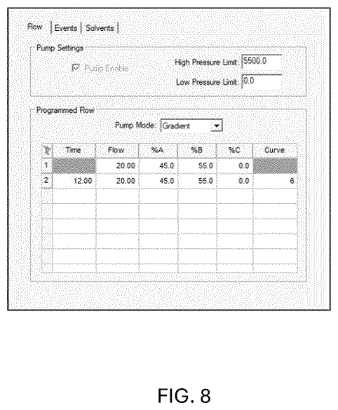

In another example, high-performance liquid chromatography (HPLC) (reverse phase C18) can be used. The HPLC purification experiments were conducted using a Waters HPLC comprised of 2707 autosampler, 2489 UV-Vis detector, and 1525 pumps. The HPLC column used was Waters SunFire® Prep C18 OBD™ 5 μm, 19×250 mm. In one example, isocractic HPLC using a gradient of 45% water-55% acetonitrile (FIG. 8) can result in having the peptide elute first. The fractions can be tested for purity by HPLC-UV or LC-MS to yield a fraction purity of greater than 98%. The fractions can be combined and lyophilized to yield the purified peptide. Alternatively, gradient HPLC (FIG. 6) can be used, in which the peptide elutes last. An exemplary gradient is shown in the following Table 1.

TABLE 1
Exemplary Gradient for Gradient HPLC

| Time (min) | Water (%) | Acetonitrile (%) |
|---|---|---|
| 0 | 67 | 33 |
| 7 | 65 | 35 |
| 23 | 55 | 45 |
| 25 | 5 | 95 |
| 27.5 | 5 | 95 |
| 27.7 | 67 | 33 |
| 30 | 67 | 33 |

Fractions can be tested for purity by HPLC-UV or LC-MS. The fraction purity can be greater than 95%, greater than 96%, greater than 97%, or greater than 98%. The fractions can be combined and lyophilized to yield the purified polypeptide.

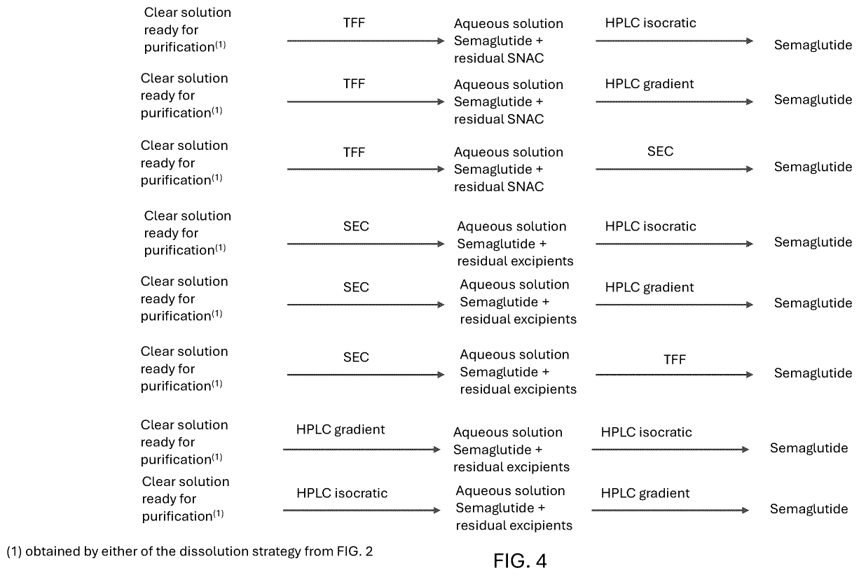

Referring to FIG. 4, the clear solution (or target filtrate) can be purified via various combinations of TFF, HPLC, and SEC to yield the polypeptide.

There are many advantages to purifying peptides through extraction instead of chemical synthesis. Some of these advantages include, but are not limited to:
a. Using two or more methods of separation can result in a higher purity peptide obtained more easily than if a peptide was chemically synthesized and then purified;
b. Using mobile phases that exclude commonly used acids (such as acetic acid and trifluoroacetic acid), the free base form of a peptide can be obtained, which may be very important for its activity and otherwise very challenging to obtain by chemical synthesis;
c. There is less risk of racemization of the target molecule compared to chemical synthesis;
d. Extraction of a peptide can be much faster than chemical synthesis;
e. Fractions with below target purity can be further processed to improve purity;
f. Extraction decreases the use of toxic organic solvents involved in peptide synthesis (such as dimethylformamide or dichloromethane); and
g. The target peptide can be collected into a final fraction or further processed into a formulation directly after purification, which eliminates one freeze drying step.

Acetic acid and trifluoroacetic acid can be important reagents used during peptide synthesis and purification. However, use of these acids can lead to acid contamination or undesirable salt forms when trying to purify a target peptide.

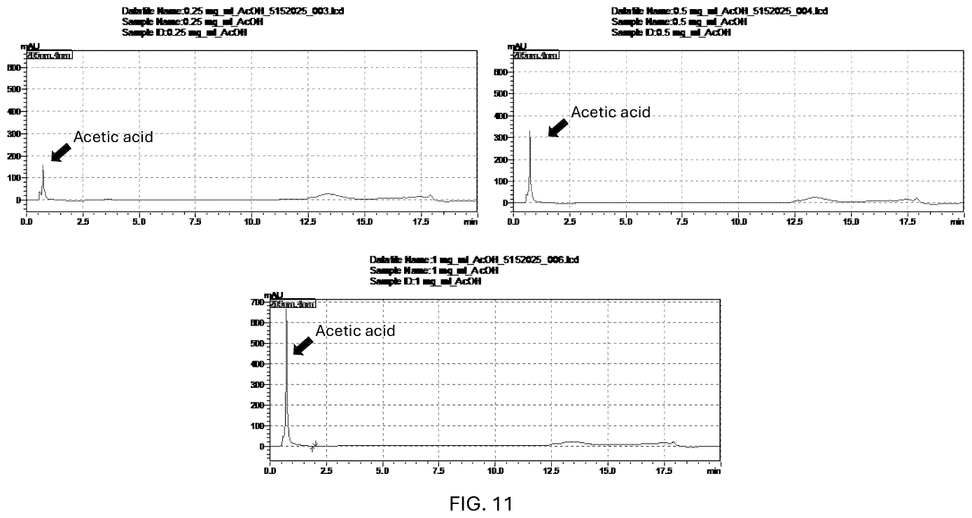

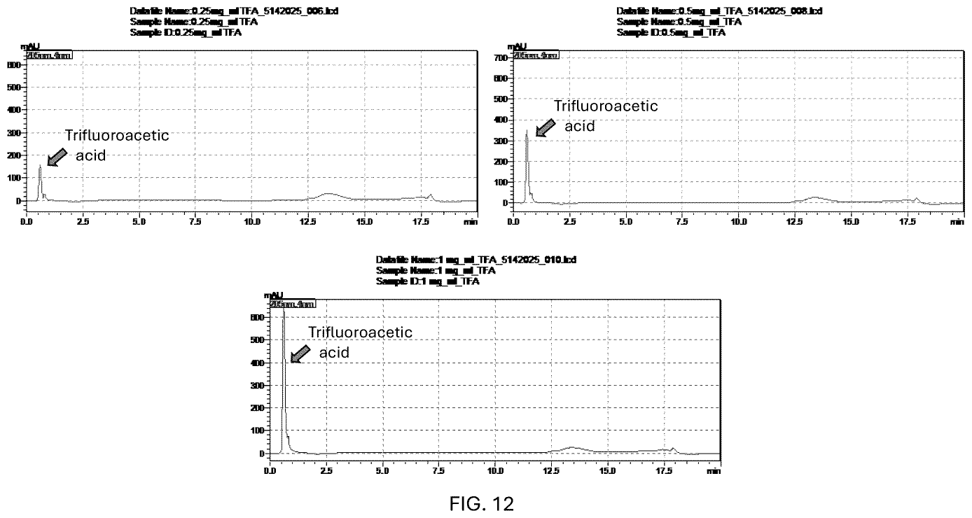

The presence of acetic acid (FIG. 11) and trifluoroacetic acid (FIG. 12) is detectable in HPLC chromatograms. The presence of acetic acid and trifluoroacetic acid was tested using Shimadzu—LC-2060C HPLC-UV, using a C18 column (Restek Raptor Inert C18; 1.8 μM, 50×2.1 mm column), with detection at 205 nm and using the following gradient.

| Time (min) | Water (%) | Acetonitrile (%) |
|---|---|---|
| 0 | 100 | 0 |
| 10 | 60 | 40 |
| 15 | 5 | 95 |
| 16 | 100 | 0 |
| 20 | 100 | 0 |

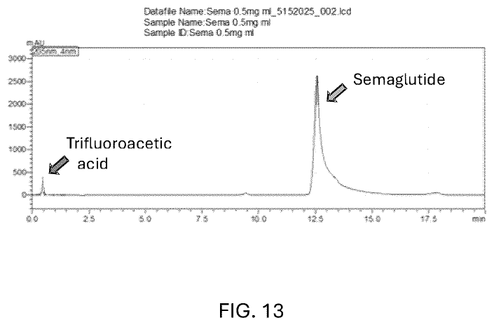

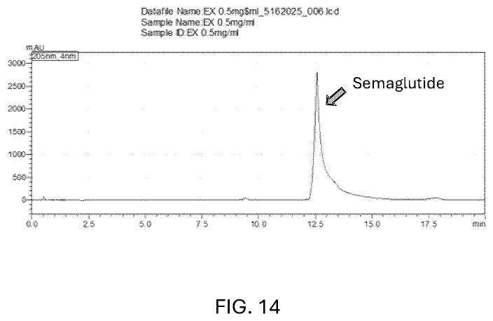

The chemical synthesis and purification of a peptide using acids can result in residual acid or the salt form of a peptide with detectable levels of said acid. For example, the chemical synthesis of semaglutide using trifluoroacetic acid results in the trifluoroacetate salt form of semaglutide (FIG. 13). This can be avoided by utilizing a purification strategy that involves extracting a peptide from a powder composition, for example the extraction and purification of semaglutide from an FDA-approved, clinically available oral tablet form of semaglutide using the methods described herein (FIG. 14). In this instance, semaglutide is purified and isolated in its salt free or free base form without the presence of acid or anions.

The salt free, free base, or free acid form of a peptide can potentially affect its pharmacological activity and have significant clinical implications. In the case of semaglutide, only the salt free or free base form is FDA approved as a medical treatment for disease. Therefore, preparations of semaglutide that contain acids from chemical synthesis or a salt form, such as the trifluoroacetate salt form of semaglutide, would require additional purification steps to achieve its salt free or free base form prior to being formulated as a clinical therapeutic. Peptide purification strategies involving extraction, such as those described herein, would therefore be ideal for generating therapeutic grade, FDA-approved semaglutide.

In some embodiments, a peptide can be extracted from a powder to purify the peptide in its salt free, free base, or free acid form using the methods described herein, without the presence of residual contaminants such as acids or a peptide salt form commonly found in the chemical synthesis and purification of peptides. In some embodiments, the peptide is extracted and purified by HPLC as described in FIGS. 3 and 4.

In some embodiments, semaglutide is the peptide extracted and purified from the powder in its salt free or free base form using the methods described herein, without the presence of residual contaminants such as acids or a peptide salt form commonly found in the chemical synthesis and purification of peptides, and the powder comprises an FDA-approved, clinically available oral tablet form of semaglutide. In some embodiments, the clinically available oral tablet form of semaglutide is crushed or pulverized. In some embodiments, the semaglutide is extracted and purified by HPLC as described in FIGS. 3 and 4.

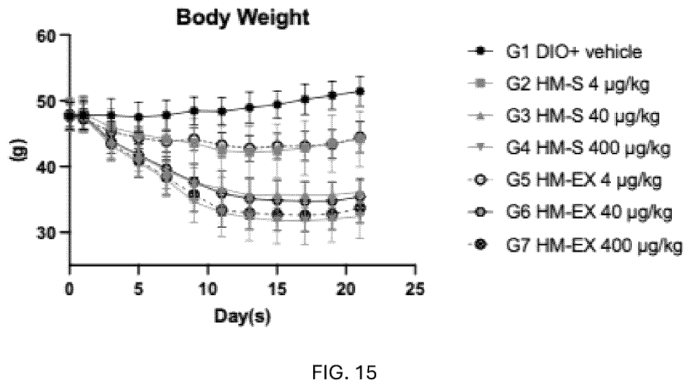

Additionally, peptides prepared through extraction purification strategies such as those described herein that produce a salt free, free base, or free acid form of a peptide can perform with the same efficacy as the active ingredient in current pharmaceutical formulations of the peptide. For example, in vivo studies in mice showed that a peptide purified through the extraction methods described herein, for instance to isolate the salt free or free base form of semaglutide from an FDA-approved, clinically available oral tablet form of semaglutide, performed equivalently to the pharmacological activity of an alternative FDA-approved, clinically available injectable formulation of semaglutide (FIGS. 15-18).

In some embodiments, the peptide extracted and purified from the powder in its salt free, free base, or free acid form using the methods described herein, without the presence of residual contaminants such as acids or a peptide salt form commonly found in the chemical synthesis and purification of peptides, is prepared and used in vivo in mammals. In some embodiments, the extracted and purified peptide performs as effectively in mammals as an alternative FDA-approved, clinically available pharmaceutical equivalent peptide that has been chemically synthesized and purified into the salt free, free base, or free acid form of the peptide. In some embodiments, the alternative FDA-approved, clinically available pharmaceutical equivalent peptide is an injectable formulation. In some embodiments, the alternative FDA-approved, clinically available pharmaceutical equivalent peptide is an injectable formulation, and the extracted and purified peptide performs as effectively in mammals as the alternative FDA-approved, clinically available pharmaceutical equivalent peptide in injectable formulation when injected into mammals. In some embodiments, the mammal is a human.

In some embodiments, semaglutide is the peptide extracted and purified from the powder in its salt free or free base form using the methods described herein, without the presence of residual contaminants such as acids or a peptide salt form commonly found in the chemical synthesis and purification of peptides; the powder comprises an FDA-approved, clinically available oral tablet form of semaglutide; and the semaglutide is prepared and used in vivo in mammals. In some embodiments, the extracted and purified semaglutide performs as effectively in mammals as an alternative FDA-approved, clinically available pharmaceutical equivalent semaglutide that has been chemically synthesized and purified into the salt free or free base form of semaglutide. In some embodiments, the alternative FDA-approved, clinically available pharmaceutical equivalent semaglutide is an injectable formulation. In some embodiments, the alternative FDA-approved, clinically available pharmaceutical equivalent semaglutide is an injectable formulation, and the extracted and purified semaglutide performs as effectively in mammals as the alternative FDA-approved, clinically available pharmaceutical equivalent semaglutide in injectable formulation when injected into mammals. In some embodiments, the mammal is a human.

### EXAMPLES

### Example 1: Strategies for Extracting Semaglutide from an FDA-Approved, Clinically Available Oral Tablet Form of Semaglutide

Several approaches were examined to extract and purify semaglutide from an FDA-approved, clinically available oral tablet form of semaglutide. The anticipated challenges were due to the multiple excipient species components of the clinically available oral tablet form of semaglutide. The composition includes:
(1) Semaglutide 7-14 mg; peptide, hydrophilic with some hydrophobic components, MW=4113;
(2) Sodium salcaprozate (SNAC) 300 mg, small molecule, MW=301;
(3) Magnesium stearate, small molecule, hydrophobic, MW=591;
(4) Povidone K-90, large polymer, water soluble, MW=1300 kDa;
(5) Cellulose crystalline, carbohydrate polymer, insoluble in water, MW>150 kDa.

The very diverse chemical class, size, hydrophilicity, and quantity of the five components of the clinically available oral tablet form of semaglutide complicated their separation. For example, a tablet contains 20 to 40 times more SNAC than semaglutide. When a component is so overwhelmingly represented, it can “bleed” through an HPLC column and/or saturate the detector, making fine separation extremely difficult and even impossible.

The very different chemical class and size of each component complicates the choice of a unique separation technique and determination of optimal purification conditions.

Moreover, the detergent-like properties of both SNAC and magnesium stearate can make their separation using a strategy based on the difference of hydrophilicity and hydrophobicity challenging.

Finally, the difference in UV absorption of all of these molecules will make their distinct detection and quantification while in mixture difficult.

Different strategies to purify semaglutide from the clinically available oral tablet form of semaglutide have been developed. These strategies can be used separately or in combination to improve their efficiency when applied to large quantities of the clinically available oral tablet form of semaglutide. A liquid extraction strategy can further enhance the process by removing a large portion of the excipients at an early stage, resulting in a more facile purification step.

Purification steps are summarized below.

### (1) Liquid Extraction

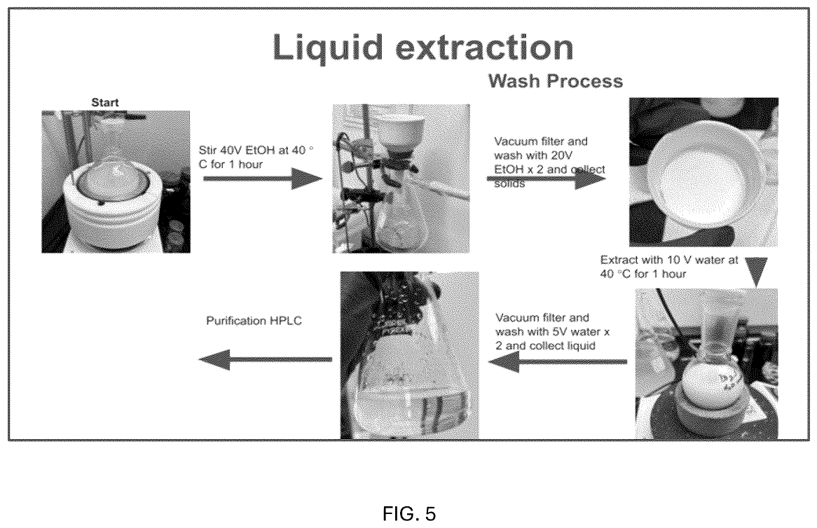

This strategy uses the different affinity of each component for water or organic solvents. First, the crushed tablet was washed with an organic solvent (preferably warmed ethanol) to remove excipient (mostly SNAC), then solubilized using water. Semaglutide was extracted from the previously obtained semi-solid and after filtration the resulting aqueous solution was ready to be purified by any of the purification strategies described below (FIG. 5). The aqueous solution contained a mixture of semaglutide and SNAC but in a more favorable proportion to promote successful purification (tablet is 1:20 semaglutide: SNAC, and the solution was about 1:10 semaglutide: SNAC).

### (2) HPLC Reverse Phase (C18) Mobile Phase H₂O/Acetonitrile Gradient

Semaglutide was purified from the clinically available oral tablet form of semaglutide using preparative HPLC. The challenge for this strategy was to develop an HPLC method which gives enough separation between SNAC and semaglutide but won't be so “slow” that the very large SNAC peak will “leak” into the semaglutide peak. After several iterations, it was determined that the gradient shown in FIG. 6 was preferred.

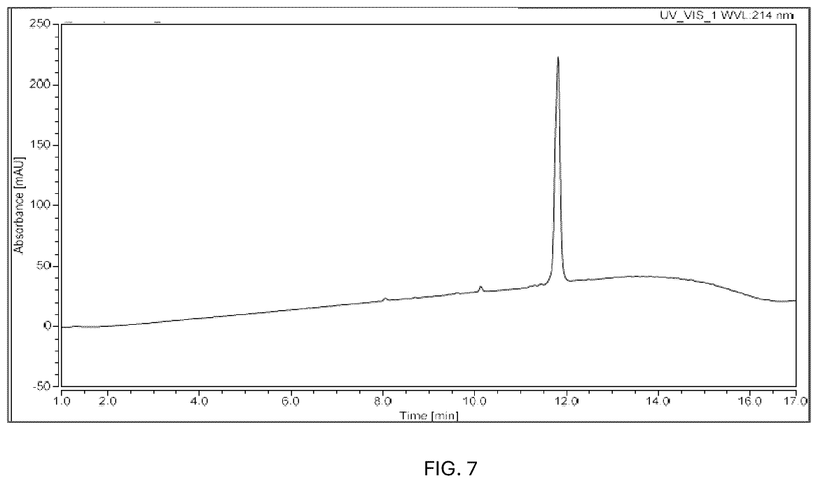

Here, 2 grams of a mixture of SNAC/semaglutide (20:1) was purified using this method as a test run. This resulted in 68 mg of 98% semaglutide (see analytical HPLC-UV, FIG. 7), which translates to a recovery yield of 71%. Purification was carried out using a Waters preparative system: Waters 2707 autosampler, Waters 2489 UV-Vis detector and Waters 1525 pumps. The column used was a Waters XSelect® Peptide, CSH C18 OBD™ Prep Column, 130 Å, 5 μM, 19 mm×250 mm. Mobile phase solvent A is water and solvent B is acetonitrile, both with no acid added. The gradient utilized is described FIG. 6 and the wavelength monitored was 214 nM. The purification resulted in an HPLC chromatogram for semaglutide shown in FIG. 7.

### (3) HPLC Reverse Phase (C18) Mobile Phase H₂O/Acetonitrile Isocratic Conditions

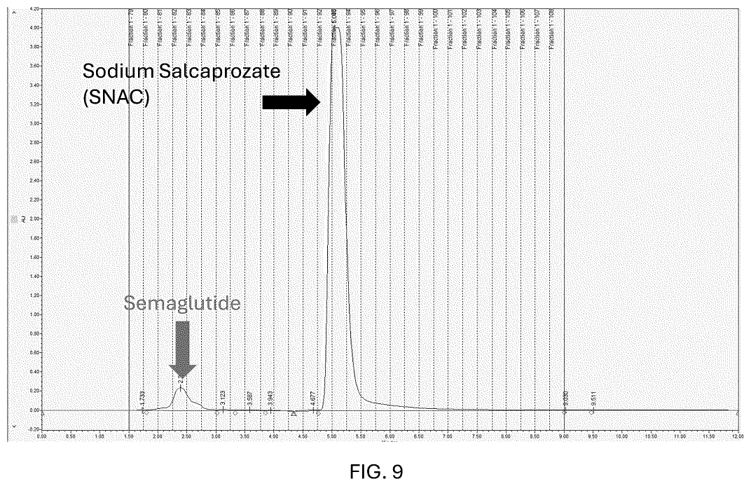

While the gradient method gives satisfactory purity, there can be concerns about the possibility that during scale up for very large purification batches, some residual SNAC could elute with semaglutide. Surprisingly, when HPLC isocratic conditions (shown in FIG. 8) were used for the purification, a switch in the order of elution of SNAC and semaglutide was observed (FIG. 9). While using a gradient, SNAC eluted first, followed approximately 5 min later by semaglutide. However, the opposite result was obtained by using isocratic conditions; semaglutide eluted first, followed approximately 3 min later by SNAC (FIG. 9). This resulted in high purity semaglutide with no concern for potential SNAC contamination, even for mixtures with very high excess SNAC. Additionally, this method was more economical as less acetonitrile was used for the purification.

### (4) Tangential Flow Filtration (TFF)

TFF uses membranes with pores allowing molecules of a specific size to go through while larger molecules are retained. To separate semaglutide from the excipients of the clinically available oral tablet form of semaglutide, the mixture can be subjected to two steps of TFF using two different membranes. First, the large polymers (cellulose and PVP) can be removed using a membrane with a 10 kDa molecular weight cut-off. The resulting filtrate only contains semaglutide and two small molecule excipients (SNAC and stearate). The filtrate can then be submitted to a second step of filtration-concentration using a membrane with a 2 kDa molecular weight cut-off. SNAC and stearate can be filtered off while semaglutide remains in the solution and then is concentrated.

Three rounds of filtration can be performed using the 2 kDa membrane. In each case, the solution can be concentrated to 1:10 of its starting volume. Water is added to have the same volume of solution as the start of the first step of filtration and concentrated to 1:10 of the volume. This strategy allows the extraction of semaglutide with high purity (purity >98%) and excellent recovery yield while only using water as a solvent.

### (5) Size Exclusion Chromatography (SEC)

Because of the significant differences in size between the components of the clinically available oral tablet form of semaglutide, SEC was also considered to be a viable approach to purify semaglutide despite usually being reserved for much larger molecules (such as antibodies or proteins) than semaglutide, SNAC, or magnesium stearate. The other two advantages of SEC are: (a) larger molecules elute faster, resulting in semaglutide eluting before SNAC; and (b) the mobile phase can be an aqueous buffer with no need for expensive acetonitrile or other organic solvents.

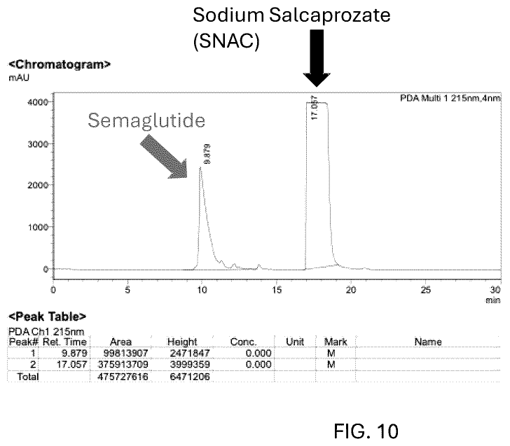

Using a mixture of SNAC and semaglutide (20:1, respectively), this strategy was demonstrated to purify semaglutide from the clinically available oral tablet form of semaglutide. FIG. 10 shows that semaglutide eluted after 9.9 min, while SNAC eluted approximately 7 min later at approximately 17 min. This separation led to the surprising understanding that SEC can be used to purify large quantities of semaglutide from the clinically available oral tablet form of semaglutide. This purification was achieved using an Xbridge Premier Protein SEC column, 250 Å, 2.7.8×5 M, 19 mm×250 mm. Mobile phase was 25 mM phosphate buffer, pH 7.00, 100 mM KCl, 10% ACN.

### (6) Combination of Two or More Extraction Strategies

The strategies described above can be combined to facilitate purification of large-scale quantities of polypeptides from complex mixtures, such as semaglutide from the clinically available oral tablet form of semaglutide. For example, TFF can be used in combination with reverse phase HPLC (isocratic).

Once the tablets are reduced into powder, warm water (40° C.) can be added to the powder and the suspension can be mixed for 1 hour. The insoluble residual molecules can be removed by filtration and the resulting solution can be submitted to TFF, first using a 10 kDa molecular weight cut-off membrane, and secondly the filtrate can be concentrated using 2 kDa molecular weight cut-off membrane. The first filtration can remove both large molecular weight polymers (PVP and cellulose), and the second filtration can remove magnesium stearate and most of the SNAC. Finally, the solution can be purified by reverse phase HPLC (isocratic) to obtain high purity semaglutide as described herein.

As noted above, there are several other combinations of purification methods that may be utilized (FIG. 4).

### Example 2: Purification of Semaglutide from an FDA-Approved, Clinically Available Oral Tablet Form of Semaglutide

FIGS. 11 and 12 show HPLC-UV chromatograms of increasing concentrations of acetic acid and trifluoroacetic acid, respectively. The presence of this peak in purified target peptide chromatograms can indicate that the target peptide fraction has acid contamination or is in a salt form. For example, FIG. 13 shows an HPLC-UV chromatogram of semaglutide that has been chemically synthesized and cleaved from its solid phase resin using trifluoroacetic acid. This results in the trifluoroacetate salt form of semaglutide, which is less desirable than the free base form of semaglutide because the free base form has been shown to be important for its pharmacological activity and is the active ingredient in approved semaglutide medications. This trifluoroacetate salt form can be avoided, and the purification of the more desirable free base form of semaglutide can be enhanced, by extracting and purifying semaglutide from powder sources such as an FDA-approved, clinically available oral tablet form of semaglutide. Using the methods described herein, FIG. 14 shows an HPLC-UV chromatogram of semaglutide extracted and purified from the clinically available oral tablet form of semaglutide that does not show any peaks corresponding to the presence of acetic acid or trifluoroacetic acid. The result is a highly pure free base form of semaglutide.

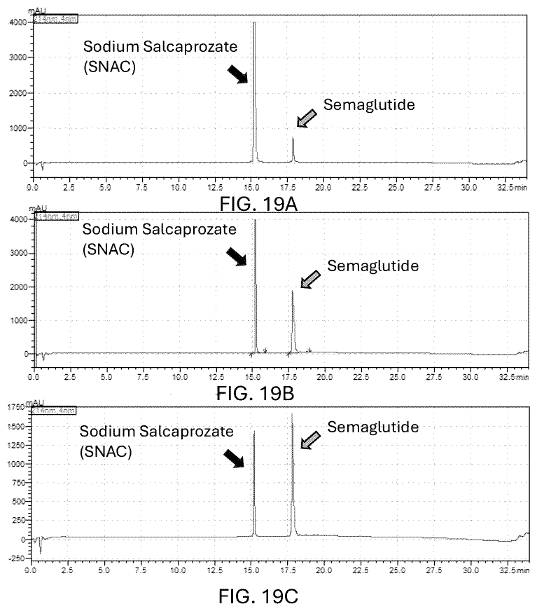

Semaglutide was extracted from FDA-approved, clinically available semaglutide tablets using a combination of TFF and preparative HPLC. Ten tablets containing semaglutide were crushed into a fine powder and suspended in 100 mL of water. The suspension was shaken for 10 minutes then filtered to obtain a clear solution containing semaglutide and SNAC (or target filtrate). A large portion of SNAC was removed using TFF. 100 mL of the solution obtained by dissolving the crushed tablets in water went through five rounds of TFF using a cassette with a molecular weight cut-off of 2 kDa. During each round, the starting solution was concentrated to 10 mL. The 10 mL of the final “concentrate” contained less SNAC than the starting solution. The concentrate was then diluted with 90 mL of “fresh” DI water and concentrated again. After each step, the “concentrated” solution was analyzed by HPLC-UV to confirm a reduction in the amount of SNAC with each TFF step (FIG. 19). After five rounds of TFF, the final concentrated solution (10 mL), was purified by HPLC, using the conditions and gradient described in FIG. 1. The fractions collected during the HPLC purification were analyzed by HPLC-UV and the fraction with a purity equal or superior to 98% were pooled and freeze dried. The semaglutide obtained was then tested for the presence or absence of acetic acid and trifluoroacetic acid before being used in in vivo experiments for weight loss activity.

The presence of acetic acid or trifluoroacetic acid was determined using a Shimadzu—LC-2060C HPLC-UV system with a C18 column (Restek Raptor Inert C18; 1.8 μM, 50×2.1 mm column). The following gradient was selected to separate the targeted organic acids which were detected at 205 nM wavelength.

| Time (min) | Water (%) | Acetonitrile (%) |
|---|---|---|
| 0 | 100 | 0 |
| 10 | 60 | 40 |
| 15 | 5 | 95 |
| 16 | 100 | 0 |
| 20 | 100 | 0 |

### Example 3: Pharmacological Effect of Powder-Extracted Semaglutide from an FDA-Approved, Clinically Available Oral Tablet Form Vs. An Alternative FDA-Approved, Clinically Available Injectable Formulation

### (1) Study Objective

This study was conducted to compare the effects of semaglutide obtained from FDA-approved, clinically available oral semaglutide tablets using the extraction and purification strategies described herein with an alternative FDA-approved, clinically available injectable formulation of semaglutide on body weight and blood glucose in diet-induced obesity (DIO) mice.

### (2) Test Articles

HM-S (an FDA-approved, clinically available injectable formulation of semaglutide) was supplied as a solution in water at a concentration of 800 μg/mL in water. HM-EX (semaglutide obtained from FDA-approved, clinically available semaglutide tablets) was supplied as a solution at a concentration of 800 μg/mL in water. HM-EX was obtained after coarse purification and fine purification by TFF, then preparative HPLC using the extraction and purification strategies described herein in Example 2. After extraction and purification, the purified semaglutide was freeze dried and then solubilized in water to obtain the indicated concentration. Both were stored at 2-8° C.

### (3) Reagents and Equipment

Isoflurane was obtained from Orbiepharm, Lot No.: 20241003, Exp: 16 Oct. 2026. Electronic scales were provided by Huazhi (Fujian) Electronic Technology Co., Ltd. PTY-B620. Small animal anesthesia machine was provided by RWD Life Science, R500IP. Centrifuge was a Thermo Sorvall Legend. Automatic biochemical analyzer was a Hitachi 3110.

### (4) Animals

Naïve male C57BL/6J mice weighing 45-55 g were acclimated for 5 days. Cages had two mice/cage by treatment group. Mice had free access to high fat diet food (irradiated, Research Diets D12492) and water. A total of 77 male C57BL/6J mice (extra 7 animals were ordered to ensure adequate number of animals for grouping) were purchased from qualified vendors. Mice were free of specific pathogens.

### (5) Allocation to Treatment Groups

Seventy DIO mice were randomly divided into 7 groups according to body weight (primary) and glucose (secondary) using Biobook software to reduce bias. The day of randomized grouping was defined as Day −3. The grouping and administration schedule are shown in Table 2.

TABLE 2
Treatment Groups

| | | | | Dose | Dose | | |
|---|---|---|---|---|---|---|---|
| | | | | Conc. | volume | Dosage | |
| Group | N | Diet | Route | (μg/mL) | (mL/kg) | (μg/kg/day) | Regimen |
| G1: Vehicle^{a} | 10 | HFD | s.c. | N/A | 5 | N/A | QD, From Day 1 |
| | | | | | | | to Day 20 |
| G2: HM-S- | 10 | HFD | s.c. | 0.8 | 5 | 4 | QD, From Day 1 |
| low | | | | μg/mL | | μg/kg/day | to Day 20 |
| G3: HM-S- | 10 | HFD | s.c. | 8 | 5 | 40 | QD, From Day 1 |
| mid | | | | μg/mL | | μg/kg/day | to Day 20 |
| G4: HM-S- | 10 | HFD | s.c. | 80 | 5 | 400 | QD, From Day 1 |
| high | | | | μg/mL | | μg/kg/day | to Day 20 |
| G5: HM-EX- | 10 | HFD | s.c. | 0.8 | 5 | 4 | QD, From Day 1 |
| low | | | | μg/mL | | μg/kg/day | to Day 20 |
| G6: HM-EX- | 10 | HFD | s.c. | 8 | 5 | 40 | QD, From Day 1 |
| mid | | | | μg/mL | | μg/kg/day | to Day 20 |
| G7: HM-EX- | 10 | HFD | s.c. | 80 | 5 | 400 | QD, From Day 1 |
| high | | | | μg/mL | | μg/kg/day | to Day 20 |

^{a}Water.

### (6) Procedure

On Day −3, mice were randomly grouped according to body weight as shown in Table 2. Mice baseline fasting glucose was measured after 6 hrs fasting. On Day 1, mice in G1-G7 were treated as shown in Table 2. Mice body weight was recorded every other day. Food intake weight was recorded every other day during the treatment period as follows: (a) food was weighed before adding it to the cage; (b) remaining food was weighed after removing it from the cage, just before replacing it. The difference between these two weights provided the total food intake for that period. On Day 21, mice fasting glucose was measured after 6 hrs fasting. Animals were then euthanized by excessive CO₂ inhalation. Animals were observed daily for their health status and general responses to treatments. There were no abnormalities in the appearance or behavior of mice in this study.

### (7) Results

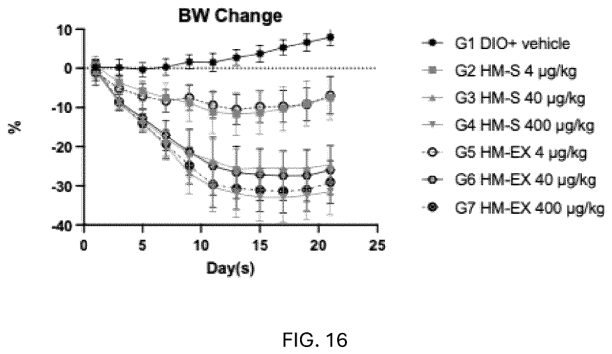

Body weight (BW) and BW change over the course of the study can be found in FIGS. 15 and 16 and Table 3. Body weight (grams) and BW change (%) of all mice were recorded every other day. Results were expressed as mean±SEM. All groups were compared with G1. Data analysis was performed by repeated measures ANOVA followed by Bonferroni's multiple comparison test.

TABLE 3
Body Weight & BW Change

| | | | Day1 | | Day3 | | Day5 | | Day7 | |
|---|---|---|---|---|---|---|---|---|---|---|
| | | Day-3 | | BW | | BW | | BW | | BW |
| Group | | BW(g) | BW(g) | change | BW(g) | change | BW(g) | change | BW(g) | change |
| G1: | Mean | 47.67 | 47.81 | 0.00 | 47.78 | 0.00 | 47.50 | 0.00 | 47.83 | 0.00 |
| Vehi- | SEM | 0.64 | 0.71 | 0.00 | 0.81 | 0.01 | 0.73 | 0.01 | 0.73 | 0.01 |
| cle | | | | | | | | | | |
| G2: | Mean | 47.70 | 48.06 | 0.01 | 45.99 | −0.04 | 44.97 | −0.06 | 44.16 | −0.08 |
| HM- | SEM | 0.74 | 0.87 | 0.01 | 0.88 | 0.01 | 0.92 | 0.01 | 1.09 | 0.01 |
| S-low | | | | | | | | | | |
| G3: | Mean | 48.02 | 47.72 | −0.01 | 43.94 | −0.08 | 41.72 | −0.13 | 39.37 | −0.18 |
| HM- | SEM | 0.77 | 0.83 | 0.01 | 0.83 | 0.01 | 0.79 | 0.01 | 0.86 | 0.01 |
| S-mid | | | | | | | | | | |
| G4: | Mean | 47.35 | 47.51 | 0.00 | 43.15 | −0.09 | 40.64 | −0.14 | 38.02 | −0.20 |
| HM- | SEM | 0.62 | 0.69 | 0.00 | 0.64 | 0.00 | 0.69 | 0.01 | 0.84 | 0.01 |
| S- | | | | | | | | | | |
| high | | | | | | | | | | |
| G5: | Mean | 47.82 | 47.24 | −0.01 | 45.30 | −0.05 | 44.35 | −0.07 | 43.79 | −0.08 |
| HM- | SEM | 0.73 | 0.49 | 0.01 | 0.50 | 0.01 | 0.53 | 0.01 | 0.49 | 0.01 |
| EX- | | | | | | | | | | |
| low | | | | | | | | | | |
| G6: | Mean | 47.91 | 47.62 | −0.01 | 43.84 | −0.08 | 41.80 | −0.13 | 39.70 | −0.17 |
| HM- | SEM | 0.59 | 0.65 | 0.01 | 0.58 | 0.01 | 0.50 | 0.01 | 0.58 | 0.01 |
| EX- | | | | | | | | | | |
| mid | | | | | | | | | | |
| G7: | Mean | 47.58 | 47.94 | 0.01 | 43.49 | −0.09 | 40.94 | −0.14 | 38.42 | −0.19 |
| HM- | SEM | 0.67 | 0.84 | 0.00 | 0.81 | 0.01 | 0.80 | 0.01 | 0.84 | 0.01 |
| EX- | | | | | | | | | | |
| high | | | | | | | | | | |

| | | Day9 | | Day11 | | Day13 | | Day15 | |
|---|---|---|---|---|---|---|---|---|---|
| | | | BW | | BW | | BW | | BW |
| Group | | BW(g) | change | BW(g) | change | BW(g) | change | BW(g) | change |
| G1: | Mean | 48.46 | 0.02 | 48.39 | 0.02 | 48.96 | 0.03 | 49.44 | 0.04 |
| Vehi- | SEM | 0.68 | 0.01 | 0.67 | 0.01 | 0.75 | 0.01 | 0.65 | 0.01 |
| cle | | | | | | | | | |
| G2: | Mean | 43.55 | −0.09 | 42.41 | −0.11 | 42.17 | −0.12 | 42.36 | −0.11 |
| HM- | SEM | 1.19 | 0.02 | 1.28 | 0.02 | 1.27 | 0.02 | 1.30 | 0.02 |
| S- | | | | | | | | | |
| low | | | | | | | | | |
| G3: | Mean | 37.54 | −0.22 | 36.67 | 0.24 | 35.62 | −0.26 | 35.73 | −0.25 |
| HM- | SEM | 0.96 | 0.02 | 0.84 | 0.02 | 0.77 | 0.02 | 0.66 | 0.01 |
| S- | | | | | | | | | |
| mid | | | | | | | | | |
| G4: | Mean | 34.63 | −0.27 | 33.00 | −0.30 | 32.23 | −0.32 | 31.81 | −0.33 |
| HM- | SEM | 1.00 | 0.02 | 1.15 | 0.02 | 1.12 | 0.02 | 1.12 | 0.02 |
| S- | | | | | | | | | |
| high | | | | | | | | | |
| G5: | Mean | 44.18 | −0.08 | 43.26 | −0.09 | 42.75 | −0.11 | 43.09 | −0.10 |
| HM- | SEM | 0.55 | 0.01 | 0.59 | 0.01 | 0.62 | 0.01 | 0.62 | 0.01 |
| EX- | | | | | | | | | |
| low | | | | | | | | | |
| G6: | Mean | 37.66 | −0.21 | 35.92 | −0.25 | 35.15 | −0.27 | 34.88 | −0.27 |
| HM- | SEM | 0.87 | 0.02 | 1.09 | 0.02 | 0.99 | 0.02 | 0.97 | 0.02 |
| EX- | | | | | | | | | |
| mid | | | | | | | | | |
| G7: | Mean | 35.75 | −0.25 | 33.42 | −0.30 | 32.94 | 0.31 | 32.74 | −0.31 |
| HM- | SEM | 0.82 | 0.01 | 0.83 | 0.02 | 0.80 | 0.0 | 0.79 | 0.02 |
| EX- | | | | | | | | | |
| high | | | | | | | | | |

| | | |
|---|---|---|
| Day17 | Day19 | Day21 |

| | | BW | | BW | | BW |
|---|---|---|---|---|---|---|
| Group | BW(g) | change | BW(g) | change | BW(g) | change |

| | | | | | | | |
|---|---|---|---|---|---|---|---|
| G1: | Mean | 48.46 | 0.02 | 48.39 | 0.02 | 48.96 | 0.03 |
| Vehi- | SEM | 0.68 | 0.01 | 0.67 | 0.01 | 0.75 | 0.01 |
| cle | | | | | | | |
| G2: | Mean | 43.55 | −0.09 | 42.41 | −0.11 | 42.17 | −0.12 |
| HM-S- | SEM | 1.19 | 0.02 | 1.28 | 0.02 | 1.27 | 0.02 |
| low | | | | | | | |
| G3: | Mean | 37.54 | −0.22 | 36.67 | −0.24 | 35.62 | −0.26 |
| HM-S- | SEM | 0.96 | 0.02 | 0.84 | 0.02 | 0.77 | 0.02 |
| mid | | | | | | | |
| G4: | Mean | 34.63 | −0.27 | 33.00 | −0.30 | 32.23 | −0.32 |
| HM-S- | SEM | 1.00 | 0.02 | 1.15 | 0.02 | 1.12 | 0.02 |
| high | | | | | | | |
| G5: | Mean | 44.18 | −0.08 | 43.26 | −0.09 | 42.75 | −0.11 |
| HM- | SEM | 0.55 | 0.01 | 0.59 | 0.01 | 0.62 | 0.01 |
| EX- | | | | | | | |
| low | | | | | | | |
| G6: | Mean | 37.66 | −0.21 | 35.92 | −0.25 | 35.15 | −0.27 |
| HM- | SEM | 0.87 | 0.02 | 1.09 | 0.02 | 0.99 | 0.02 |
| EX- | | | | | | | |
| mid | | | | | | | |
| G7: | Mean | 35.75 | −0.25 | 33.42 | −0.30 | 32.94 | −0.31 |
| HM- | SEM | 0.82 | 0.01 | 0.83 | 0.02 | 0.80 | 0.02 |
| EX- | | | | | | | |
| high | | | | | | | |

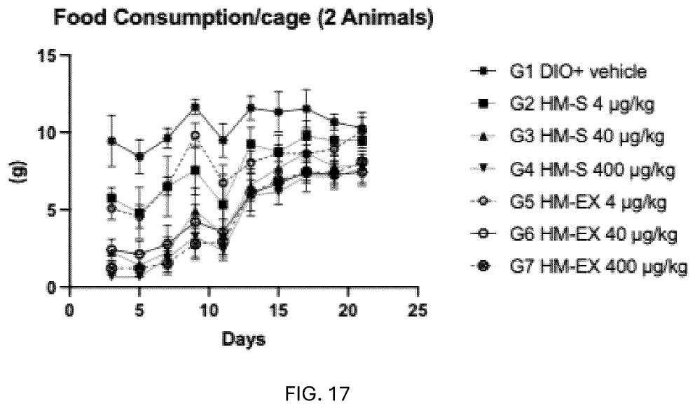

Food intake per cage over the course of the study can be found in FIG. 17 and Table 4. Food intake per cage (grams; 2 mice/cage) was recorded every other day. Results were expressed as mean±SEM. All groups were compared with G1. Data analysis was performed by repeated measures ANOVA followed by Bonferroni's multiple comparison test.

TABLE 4
Food Intake/Each Cage

| | | Food intake/cage/2 animals (g) | | | | | | | | | |
|---|---|---|---|---|---|---|---|---|---|---|---|
| Group | | Day3 | Day5 | Day7 | Day9 | Day11 | Day13 | Day15 | Day17 | Day19 | Day21 |
| G1: | Mean | 9.44 | 8.44 | 9.62 | 11.64 | 9.48 | 11.58 | 11.32 | 11.53 | 10.67 | 10.27 |
| Vehicle | SEM | 0.74 | 0.49 | 0.29 | 0.23 | 0.49 | 0.35 | 0.58 | 0.56 | 0.23 | 0.47 |
| G2: HM- | Mean | 5.76 | 4.78 | 6.50 | 7.54 | 5.33 | 9.24 | 8.73 | 9.77 | 9.47 | 9.51 |
| S-low | SEM | 0.30 | 0.75 | 0.87 | 0.71 | 0.87 | 0.47 | 0.48 | 0.45 | 0.42 | 0.23 |
| G3: HM- | Mean | 2.26 | 1.41 | 2.22 | 4.94 | 3.42 | 6.56 | 7.69 | 8.67 | 7.78 | 7.84 |
| S-mid | SEM | 0.08 | 0.33 | 0.45 | 0.65 | 0.75 | 0.37 | 0.49 | 0.65 | 0.32 | 0.22 |
| G4: HM- | Mean | 0.65 | 0.64 | 1.85 | 3.25 | 2.35 | 5.93 | 6.14 | 7.19 | 7.15 | 7.66 |
| S-high | SEM | 0.09 | 0.11 | 0.40 | 0.60 | 0.18 | 0.45 | 0.36 | 0.14 | 0.36 | 0.50 |
| G5: HM- | Mean | 5.09 | 4.56 | 6.52 | 9.81 | 6.73 | 8.03 | 8.66 | 8.62 | 8.92 | 10.08 |
| EX-low | SEM | 0.33 | 0.33 | 0.26 | 0.36 | 0.52 | 0.33 | 0.25 | 0.25 | 0.38 | 0.40 |
| G6: HM- | Mean | 2.40 | 2.12 | 2.75 | 4.20 | 3.57 | 6.02 | 6.73 | 7.38 | 7.24 | 7.42 |
| EX-mid | SEM | 0.31 | 0.38 | 0.56 | 0.50 | 0.36 | 0.19 | 0.27 | 0.55 | 0.23 | 0.31 |
| G7: HM- | Mean | 1.20 | 1.22 | 1.54 | 2.78 | 2.92 | 6.12 | 6.90 | 7.45 | 7.35 | 8.09 |
| EX-high | SEM | 0.19 | 0.13 | 0.34 | 0.46 | 0.36 | 0.69 | 0.26 | 0.33 | 0.36 | 0.22 |

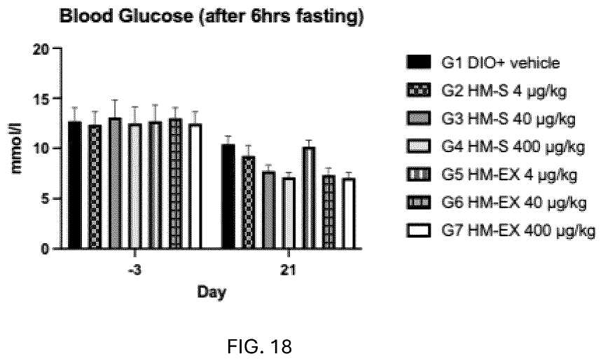

Blood glucose over the course of the study can be found in FIG. 18 and Table 5. Glucose (mmol/L) of all mice was measured at baseline and the termination day. Results were expressed as mean±SEM. All groups were compared with G1. Data analysis was performed by one-way ANOVA followed by Dunnett's multiple comparison test.

TABLE 5
Blood Glucose (after 6 hrs fasting)

Glucose
(mmol/L)

| | | |
|---|---|---|
| Group | Day 3 | Day 5 |

| | | | |
|---|---|---|---|
| G1: Vehicle | Mean | 12.73 | 10.43 |
| | SEM | 0.42 | 0.25 |
| G2: HM-S-low | Mean | 12.37 | 9.25 |
| | SEM | 0.42 | 0.33 |
| G3: HM-S-mid | Mean | 13.11 | 7.74 |
| | SEM | 0.54 | 0.19 |
| G4: HM-S-high | Mean | 12.51 | 7.14 |
| | SEM | 0.52 | 0.14 |
| G5: HM-EX-low | Mean | 12.72 | 10.16 |
| | SEM | 0.51 | 0.20 |
| G6: HM-EX-mid | Mean | 13.02 | 7.35 |
| | SEM | 0.33 | 0.22 |
| G7: HM-EX-high | Mean | 12.49 | 7.08 |
| | SEM | 0.37 | 0.17 |

### (8) Conclusion

In a 21-day study, DIO mice were treated subcutaneously once daily, starting at an average body weight of approximately 47 g. Both HM-S and HM-EX demonstrated dose-dependent effects in reducing body weight, suppressing food intake, and lowering terminal glucose levels. The two test articles exhibited comparable efficacy across low, medium and high doses. For HM-S, reductions in body weight from baseline were 32%, 26%, and 12% at doses of 400, 40, and 4 μg/kg, respectively. Similarly, HM-EX showed reductions of 31%, 27%, and 11% at the same corresponding doses.

### (9) Statistics

Data were presented as mean±SEM. Statistical analyses were performed using GraphPad Prism or SPSS. P<0.05 was considered significant.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed is:

1. A method for purifying a polypeptide comprising:
suspending a powder including the polypeptide and one or more of a matrix polymer, a carboxylate salt, and a cellulose in a polar liquid;
filtering the suspension to yield a target filtrate;
purifying the target filtrate by one or more of tangential flow filtration, size exclusion chromatography, reverse phase liquid chromatography, or combinations thereof; and
collecting the purified polypeptide.

2. The method of claim 1, wherein the polar liquid includes water, methanol, ethanol, a propanol, or a butanol.

3. The method of claim 1, wherein the polar liquid is ethanol and further comprising suspending an intermediate residue in water to form a second suspension and filtering the second suspension to produce the target filtrate.

4. The method of claim 1, wherein the polar liquid is water.

5. The method of claim 1, wherein purifying the target filtrate includes tangential flow filtration to yield a residual solution.

6. The method of claim 5, wherein the residual solution is further purified by high-performance liquid chromatography (HPLC).

7. The method of claim 6, wherein the high-performance liquid chromatography is isocratic HPLC.

8. The method of claim 6, wherein the high-performance liquid chromatography is gradient HPLC.

9. The method of claim 5, wherein the residual solution is further purified by size exclusion chromatography.

10. The method of claim 1, wherein purifying the target filtrate includes size exclusion chromatography to yield a residual solution.

11. The method of claim 10, wherein the residual solution is further purified by high-performance liquid chromatography (HPLC).

12. The method of claim 11, wherein the high-performance liquid chromatography is isocratic HPLC.

13. The method of claim 11, wherein the high-performance liquid chromatography is gradient HPLC.

14. The method of claim 1, wherein purifying the target filtrate includes isocratic HPLC to yield a residual solution.

15. The method of claim 14, wherein the residual solution is further purified by gradient HPLC.

16. The method of claim 1, wherein the powder includes an absorption enhancer.

17. The method of claim 1, wherein purifying the target filtrate includes gradient HPLC to yield a residual solution.

18. The method of claim 17, wherein the residual solution is further purified by isocratic HPLC.

19. The method of claim 1, wherein the polypeptide is semaglutide.

20. The method of claim 1, wherein the absorption enhancer is sodium 8-(2-hydroxybenzamido)octanoate.

21. The method of claim 1, wherein the matrix polymer includes polyvinylpyrrolidone.

22. The method of claim 1, wherein the cellulose includes microcrystalline cellulose.

23. The method of claim 1, wherein the carboxylate salt includes magnesium stearate.

24. The method of claim 1, wherein the powder is a crushed or pulverized tablet.

25. The method of claim 1, wherein the purified polypeptide is in its free base form.

26. The method of claim 1, wherein the purified polypeptide is in a salt free form.

27. The method of claim 1, wherein the purified polypeptide is in its free acid form.
